(19) United States
(12) Patent Application Publication
(10) Pub. No.: US 2020/0009077 A1
(43) Pub. Date: Jan. 9, 2020

(54) COMPOSITIONS FOR TREATING DERMATOLOGICAL DISEASES

(71) Applicant: J. Phillip KENNEDY, Grand Rapids, MI (US)

(72) Inventor: J. Phillip KENNEDY, Grand Rapids, MI (US)

(73) Assignee: TRUETIVA, Inc., Grand Rapids, MI (US)

(21) Appl. No.: 16/502,189

(22) Filed: Jul. 3, 2019

Related U.S. Application Data
(60) Provisional application No. 62/693,703, filed on Jul. 3, 2018.
(60) Provisional application No. 62/702,936, filed on Jul. 25, 2018.
(60) Provisional application No. 62/804,240, filed on Feb. 12, 2019.

(51) Int. Cl. A61K 31/05

(52) U.S. Cl. CPC A61K 31/05

(57) ABSTRACT

Embodiments of the invention are directed to compositions containing cannabinoid, cannabidiol, cannabidiol isomer, or cannabidiol analog and combinations thereof for treating dermatological disease, and methods for treating dermatological diseases by administering compositions containing cannabinoid, cannabidiol, or cannabidiol analog to the skin of a patient in need of treatment.

### CROSS-REFERENCE TO RELATED APPLICATIONS

This application claims priority from U.S. Provisional No. 62/693,703 entitled “Compositions for Treating Dermatological Diseases,” filed on Jul. 3, 2018, U.S. Provisional No. 62/702,936 entitled “Compositions for Treating Dermatological Diseases,” filed Jul. 25, 2018, and U.S. Provisional No. 62/804,240 entitled “Compositions for Enhanced Bioavailability and Methods for Same” filed on Feb. 12, 2019, each of which are hereby incorporated by reference in their entireties.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Not applicable

### SUMMARY OF THE INVENTION

Various embodiments are directed to compositions including a cannabinoid having a concentration of about 0.5% (w/w) to about 50% (w/w), relative to the total amount of the composition and a pharmaceutically acceptable carrier, excipient, diluent, reagent, or combinations thereof. In some embodiments, the cannabinoid may have a concentration of about 20% (w/w) to about 1% (w/w) cannabidiol, relative to the total amount of the composition, and in various embodiments, the cannabinoid may be cannabidiol, cannabidiol isomer, cannabidiol analog, or combinations thereof.

In some embodiments, the composition may further include a bioenhancer having a concentration of about 0.05% wt to about 20% wt, relative to the total weight of the composition. The bioenhancer may be a P-glycoprotein inhibitor such as, but not limited to, piperine, quercetin, genistein, naringin, sinomenine, glycyrrhizin, nitrile glycosides cuminum cyminum, zingiver officinale, lysergol, allium sativum, aloe vera, liposomes, microspheres, nanoparticles, transferosomes, ethosomes, nanoemulsions, microemulsions, lipid based systems, polymeric micelle formulations, ketoprofen-loaded solid lipid nanoparticles, and the like, which can be made from beeswax, carnauba wax, or other natural waxes, solid lipids, Ginkgo biloba, lipid-based systems, silybin lipid-based systems, ginseng lipid-based systems, hawthorn lipid-based systems, quercetin lipid-based systems, curcumin lipid-based systems, capsaicin transferosomes, colchicine tranferosomes, vincristine tranferosomes, and the like and combinations thereof.

In some embodiments, the composition may further include a humectant such as calamine, dodecylsulphate, sodium lauryl sulphate (SLS), a polyoxyethylene ester of polysorbitan, such as monooleate, monolaurate, monopalmitate, monostearate esters, esters of sorbitan, polyoxyethylenes ethers, sodium dioctylsulphosuccinate (DOSS), lecithin, sodium docusate, and the like and combinations thereof. In some embodiments, the humectant may have a concentration of about 0.01 wt. % to about 5 wt. %, relative to the total weight of the composition. In some embodiments, the composition may further include a steroid, anti-inflammatory, immune checkpoint blockade inhibitor, antibiotic, antiseptic, anti-acne agent, UV-absorbing compound, analgesic agent, or an antiviral compound, or combinations thereof, and the steroid, anti-inflammatory, immune checkpoint blockade inhibitor, antibiotic, antiseptic, anti-acne agent, UV-absorbing compound, analgesic agent, or antiviral compound, or combinations thereof may have a concentration of from about 0.01% to about 50% (wt/wt), relative to the total amount of the composition.

In certain embodiments, the composition may be in the form of a cream, lotion, salve, liniment, ointment, gel, paste, tonic, tincture, unguent, soap, shampoo, topical, oral, pills, tablet, capsule, lip balm, and the like and combinations thereof.

Other embodiments include methods for treating a dermatological disease, by administering a composition containing a cannabinoid having a of about 0.5% (w/w) to about 50% (w/w) to a patient in need of treatment. In some embodiments, the dermatological disease may be dermatitis or scarring. the concentration of the cannabinoid has a concentration in the range of about 1% to about 30% (w/w), relative to the total amount of the composition.

In some embodiments, the cannabinoid may have a concentration of about 20% (w/w) to about 1% (w/w) cannabidiol, relative to the total amount of the composition, and in various embodiments, the cannabinoid may be cannabidiol, cannabidiol isomer, cannabidiol analog, or combinations thereof.

In some embodiments, the composition may further include a bioenhancer having a concentration of about 0.05% wt to about 20% wt, relative to the total weight of the composition. The bioenhancer may be a P-glycoprotein inhibitor such as, but not limited to, piperine, quercetin, genistein, naringin, sinomenine, glycyrrhizin, nitrile glycosides cuminum cyminum, zingiver officinale, lysergol, allium sativum, aloe vera, liposomes, microspheres, nanoparticles, transferosomes, ethosomes, nanoemulsions, microemulsions, lipid based systems, polymeric micelle formulations, ketoprofen-loaded solid lipid nanoparticles, and the like, which can be made from beeswax, carnauba wax, or other natural waxes, solid lipids, Ginkgo biloba, lipid-based systems, silybin lipid-based systems, ginseng lipid-based systems, hawthorn lipid-based systems, quercetin lipid-based systems, curcumin lipid-based systems, capsaicin transferosomes, colchicine tranferosomes, vincristine tranferosomes, and the like and combinations thereof.

In some embodiments, the composition may include a steroid, humectant anti-inflammatory, immune checkpoint blockade inhibitor, antibiotic, antiseptic, anti-acne agent, UV-absorbing compound, analgesic agent, or an antiviral compound, or combinations thereof.

### DESCRIPTION OF THE DRAWINGS

The patent or application file contains at least one drawing executed in color. Copies of this patent or patent application publication with color drawing(s) will be provided by the Office upon request and payment of the necessary fee.

Examples of the specific embodiments are illustrated in the accompanying drawings. While the invention will be described in conjunction with these specific embodiments, it will be understood that it is not intended to limit the invention to such specific embodiments. On the contrary, it is intended to cover alternatives, modifications, and equivalents as may be included within the spirit and scope of the invention. In the following description, numerous specific details are set forth in order to provide a thorough understanding of the present invention. The present invention may be practiced without some or all of these specific details. In other instances, well known process operations have not been described in details so as to not unnecessarily obscure the present invention.

FIG. 1 is a comparison of the structure of a PD-L1 dimer bound by BMS-202 (left panel) and cannabidiol (right panel).

FIG. 2 is a photograph showing the hands of a dermatitis patient before treatment with a composition of the invention.

FIG. 3 is a photograph showing the hands of a dermatitis patient in FIG. 1 after 30 days of topical treatment with a composition of the invention.

FIG. 4 is a photograph showing a patient with seborrheic dermatitis before treatment with a composition of the invention.

FIG. 5 is a photograph showing the patient of FIG. 3 after 1 week of topical treatment with a composition of the invention.

FIG. 6 is a photograph showing the palms of a patient after surgery and before treatment with a composition of the invention.

FIG. 7 is a photograph showing the palms of the patient in FIG. 5 after 4-weeks of topical treatment with a composition of the invention on the left hand.

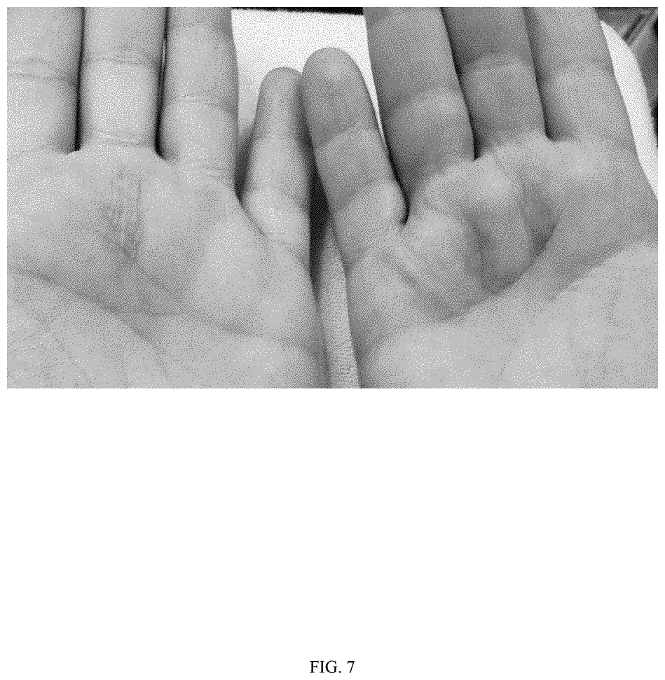

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 μm to 8 μm is stated, 2 μm, 3 μm, 4 μm, 5 μm, 6 μm, and 7 μm are also intended to be explicitly disclosed, as well as the range of values greater than or equal to 1 μm and the range of values less than or equal to 8 μm.

All percentages, parts and ratios are based upon the total weight of the compositions and all measurements made are at about 25° C., unless otherwise specified.

The singular forms “a,” “an,” and “the” include plural referents unless the context clearly dictates otherwise. Thus, for example, reference to a “polymer” includes a single polymer as well as two or more of the same or different polymers; reference to an “excipient” includes a single excipient as well as two or more of the same or different excipients, and the like.

The word “about” when immediately preceding a numerical value means a range of plus or minus 10% of that value, e.g, “about 50” means 45 to 55, “about 25,000” means 22,500 to 27,500, etc, unless the context of the disclosure indicates otherwise, or is inconsistent with such an interpretation. For example, in a list of numerical values such as “about 49, about 50, about 55, “about 50” means a range extending to less than half the interval(s) between the preceding and subsequent values, e.g, more than 49.5 to less than 52.5. Furthermore, the phrases “less than about” a value or “greater than about” a value should be understood in view of the definition of the term “about” provided herein.

The terms “administer,” “administering” or “administration” as used herein refer to either directly administering a compound (also referred to as an agent of interest) or pharmaceutically acceptable salt of the compound (agent of interest) or a composition to a subject.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical, cosmetic or other agent across a tissue layer such as the stratum corneum or stratum spinosum.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. In embodiments or claims where the term comprising is used as the transition phrase, such embodiments can also be envisioned with replacement of the term “comprising” with the terms “consisting of” or “consisting essentially of.”

The term “disorder” is used in this disclosure to mean, and is used interchangeably with the terms disease, condition, syndrome, or illness, unless otherwise indicated.

The terms “effective amount” and “therapeutically effective amount” are used interchangeably in this disclosure and refer to an amount of a compound that, when administered to a subject, is capable of reducing a symptom of a disorder in a subject or enhance the texture, appearance, color, sensation, or hydration of the intended tissue treatment area. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” or “cosmetically acceptable” is employed herein to refer to those agents of interest/compounds, salts, compositions, dosage forms, etc, which are within the scope of sound medical judgment—suitable for use in contact with the tissues of human beings and/or other mammals without excessive toxicity, irritation, allergic response, or other problem or complication, commensurate with a reasonable benefit/risk ratio. In some aspects, pharmaceutically acceptable means approved by a regulatory agency of the federal or a state government, or listed in the U.S. Pharmacopeia or other generally recognized pharmacopeia for use in mammals (e.g, animals), and more particularly, in humans.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Non-limiting examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “patient” and “subject” are interchangeable and may be taken to mean any living organism which may be treated with compounds of the present invention. As such, the terms “patient” and “subject” may include, but is not limited to, any non-human mammal, primate or human. In some embodiments, the “patient” or “subject” is a mammal, such as mice, rats, other rodents, rabbits, dogs, cats, swine, cattle, sheep, horses, primates, or humans. In some embodiments, the patient or subject is an adult, child or infant. In some embodiments, the patient or subject is a human.

The term “treating” is used herein, for instance, in reference to methods of treating a skin disorder or a systemic condition, and generally includes the administration of a compound or composition which reduces the frequency of, or delays the onset of, symptoms of a medical condition or enhance the texture, appearance, color, sensation, or hydration of the intended tissue treatment area of the tissue surface in a subject relative to a subject not receiving the compound or composition. This can include reversing, reducing, or arresting the symptoms, clinical signs, and underlying pathology of a condition in a manner to improve or stabilize a subject's condition.

By hereby reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by hereby reserving the right to proviso out or exclude any individual substituents, analogs, compounds, ligands, structures, or groups thereof, or any members of a claimed group, less than the full measure of this disclosure can be claimed for any reason. Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Various embodiments are directed compositions and methods for treating a dermatological disease or disorder. Such compositions may include a cannabinoid and the methods may include administering a composition containing cannabinoids to a patient in need of treatment. In particular embodiments, the dermatological disease or disorder may be dermatitis.

The cannabinoids of such embodiments include any of a broad class of compounds that are known to interact with cannabinoid receptors, and encompass endocannabinoids (produced naturally in the body by animals), the phytocannabinoids (found in cannabis and some other plants), and synthetic cannabinoids (manufactured artificially). Example cannabinoids include, but are not limited to, tetrahydropyran analogs, such as, Δ⁹-tetrahydrocannabinol, Δ⁸-tetrahydrocannabinol, 6,6,9-trimethyl-3-pentyl-6H-dibenzo[b, d]pyran-1-ol, 3-(1,1-dimethylheptyl)-6,6a7,8,10,10a-hexahydro-1-1hydroxy-6,6-dimethyl-9H-dibenzo[b, d]pyran-9-ol, (−)-(3S,4S)-7-hydroxy-delta-6-tetrahydrocannabinol-1,1-dimethylheptyl, (+)-(3S,4S)-7-hydroxy-A-6-tetrahydrocannabinol, and Δ⁸-tetrahydrocannabinol-11-oic acid, piperidine analogs, such as, (−)-(6S,6aR,9R,10aR)-5,6,6a,7,8,9,10,10a-octahydro-6-methyl-1-3-[(R)-1-methyl-4-phenylbutox y]-1,9 phenanthridinediol 1-acetate), aminoalkylindole analogs, such as, (R)-(+)-[2,3-dihydro-5-methyl-3-(4-morpholinylm-ethyl)-pyrrolo[1,2,3,-de]-1,4-benzoxazin-6-yl]-1-naphthelenyl-methanone, open pyran-ring analogs, such as, 2-[3-methyl-6-(1-methylethenyl-2-cyclohexen-1-yl]-5-pentyl-1,3-benzendi-ol, and 4-(1,1-dimethylheptyl)-2,3′-dihydroxy-6′-a-(3-hydroxypropyl)-1′,-2′,3′,4′,5′,6′-hexahydrobiphen yl, lipophilic alkylamides, such as, dodeca-2E,4E,8Z,10E/Z-tetraenoic-acid-isobutylamide, cannabinoid mimetics, salts, solvates, metabolites, and metabolic precursors of these compounds and combinations thereof. In some embodiments, the cannabinoids may be derived plants including hemp, Echinacea purpurea, Echinacea angustifolia, Acmella oleracea, Helichrysum umbraculigerum, Radula marginata, and combinations thereof and oils made from these plants, and in other embodiments, the cannabinoids may be manufactured or chemically synthesized.

The compositions of various embodiments can include any number of cannabinoids in various concentrations; however, in certain embodiments, the cannabinoid may be cannabidiol (2-(6-isopropenyl-3-methyl-5-cyclohexen-1-yl)-5-pentyl-1,3-benzenediol). Cannabidiol has 7 double bonds and 30 stereoisomers. Embodiments include compositions containing each stereoisomer individually and compositions containing a combination of these stereoisomers. In particular embodiments, the compositions used in the methods of embodiments and the compositions of embodiments may include high concentrations of cannabidiol. For example, in some embodiments, cannabidiol may be about 30 w/v % to about 100 w/v % of the cannabinoids in the composition, and in other embodiments cannabidiol may be about 50 w/v % to about 100 w/v %, about 75 w/v % to about 100 w/v %, about 80 w/v % to about 100 w/v %, about 90 w/v % to about 100 w/v % of the cannabinoids in the composition.

Cannabidiol can be obtained by cold-pressing industrial hemp with trace amounts of THC. Cannabidiol in this present invention is provided as a natural constituent of hemp oil.

In some embodiments, the cannabinoids in the composition may be cannabidiol analogs. The term “cannabidiol analogs” refers to synthetically produced compounds that are structurally similar, but not structurally identical, to cannabidiol. Various cannabidiol analogs are known in the art and embodiments encompass such cannabidiol analogs. For example, PCT Publication WO2017/132526 and U.S. Pat. No. 6,630,507, which are each hereby incorporated by reference in their entireties, describes various analogs of cannabidiol. In some embodiments, the analogs of cannabidiol may be of general Formula I:

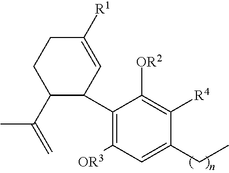

where R¹ is hydrogen, methyl, linear or branched C₂-C₁₀ alkyl, linear or branched C₂-C₁₀ alkenyl, linear or branched C₂-C₁₀ substituted alkyl, linear or branched C₂-C₁₀ substituted alkenyl, R² and R³ are each, individually, hydrogen, methyl, linear or branched C₂-C₁₀ alkyl, linear or branched C₂-C₁₀ substituted alkyl, linear or branched C₂-C₁₀ alkenyl, linear or branched C₂-C₁₀ substituted alkenyl, linear or branched C₂-C₁₀ acyl, linear or branched C₂-C₁₀ substituted acyl, an amine or amino acid, amino acid ester, R⁴ is hydrogen, substituted or unsubstituted alkyl, carboxyl, alkoxy, aryl, aryloxy, arylalkyl, halo or amino, and n may an integer of 2 to 10 and the like and salts and solvates thereof. In some embodiments, R² and R³ may, independently, be a linear or branched, substituted or unsubstituted C₂-C₁₀ acyl having a carboxylic acid terminus thereby producing a dicarboxylic acid, and salts thereof. Like cannabidiol, cannabidiol analogs can have various isomers. Embodiments include all isomers of the such cannabidiol analogs.

In some embodiments, cannabidiol analogs, such as those described above may be combined with cannabidiol, to produce a mixture of cannabidiol and cannabidiol analogs. Thus, as used herein the term “cannabidiol” encompasses cannabidiol, cannabidiol analogs, and the various isomers of cannabidiol and cannabidiol analogs.

The compositions of various embodiments can include up to about 50% (w/w) cannabidiol, cannabidiol analogs, isomers of cannabidiols, and combinations thereof (collectively, “cannabidiol”), relative to the total amount of the composition, and in some embodiments, the compositions may include from about 100% to about 0.5% (w/w) cannabidiol, relative to the total amount of the composition, 50% (w/w) to about 0.5% (w/w) cannabidiol, relative to the total amount of the composition, about 30% (w/w) to about 1% (w/w) cannabidiol, relative to the total amount of the composition, about 20% (w/w) to about 1% (w/w) cannabidiol, relative to the total amount of the composition, about 20% (w/w) to about 5% (w/w) cannabidiol, relative to the total amount of the composition, or any range or individual concentration encompassed by these example ranges. In particular embodiments, the composition may include about 15% (w/w) to about 10% (w/w) cannabidiol, relative to the total amount of the composition, or any range or individual concentration encompassed by this example range.

In certain embodiments, the cannabidiol of embodiments described above may be cannabidolic acid (“CBDA”). Without wishing to be bound by theory, CBDA may exhibit improved hydrophilicity over other isomers of cannabidiol, which may allow for improved solubility and delivery of CBDA to the skin. The CBDA may be modified, partially digested, or otherwise acted upon by enzymes in the skin to produce for example cannabidiol (CBD), which may be the active form cannabidiol in the composition. Thus, CBDA may act as a prodrug in some embodiments of the invention. Other cannabidiol analogs or isomers may produce a similar effect and are encompassed by prodrug embodiments of the invention.

The cannabidiol in the compositions of embodiments of the invention may be 100% cannabidiol, or oils, solvents, and emulsions containing cannabidiol. For example, in some embodiments, the compositions of the invention may include cannabidiol derived from hempseed oil. Hempseed oil is generally manufactured from varieties of Cannabis sativa that do not contain significant amounts of tetrahydrocannabinol (THC), the psychoactive element of the cannabis plant. This manufacturing process typically includes cleaning the seed to 99.99% before pressing the oil. Hempseed oil generally also contains omega-6 and omega-3 fatty acids. For example, about 30-35% of the weight of hempseed oil are essential fatty acids (EFAs), i.e., linoleic acid, omega-6 (LA, 55%), a-linolenic acid, omega-3 (ALA, 22%), y-linolenic acid, omega-6 (GLA, 1-4%), and stearidonic acid, omega-3 (SDA, 0-2%). Thus, the compositions of some embodiments may contain fatty acids such as omega-6 and omega-3 fatty acids.

Oils include cannabidiol oil and various plant derived oils containing cannabidiol, such as, hempseed oil, Echinacea purpurea, Echinacea angustifolia, Acmella oleracea, Helichrysum umbraculigerum, Radula marginata, and the like. In some embodiments, cannabidiol isolated from such plants or made synthetically may be formulated with an oil such as, for example, olive oil, grapeseed oil, tea tree oil, almond oil, avocado oil, sesame oil, evening primrose oil, sunflower oil, kukui nut oil, jojoba oil, walnut oil, peanut oil, pecan oil, macadamia nut oil, coconut oil, and the like and combinations thereof.

Unless indicated otherwise, the term “therapeutically effective amount” is not particularly limited, so long as the cannabinoid is present in an amount effective for treating the dermatological disease. The therapeutically effective amount of cannabinoid can be from about 2 milligrams per kilogram (mg/kg) to about 100 mg/kg, about 2 mg/kg to about 50 mg/kg, about 2 mg/kg to about 25 mg/kg, or any range or individual concentration encompassed by these example ranges, wherein mg refers to the mass or weight of the cannabinoids and kg refers to the mass or weight of the patient in need of treatment. In certain embodiments, a therapeutically effective amount of THC and/or CBD in the composition may be about 2 mg/kg to about 10 mg/kg or any range or individual concentration encompassed by these example ranges, wherein mg refers to the mass or weight of the cannabinoids and kg refers to the mass or weight of the patient in need of treatment.

In some embodiments, the dermatological disease may be dermatitis. The term “dermatitis” is meant to encompass all forms of dermatitis known in the art including: Contact dermatitis, allergic contact dermatitis, and irritant contact dermatitis. Contact dermatitis typically causes a pink or red itchy rash. Allergic contact dermatitis is a skin allergy to something that touches the skin, even if only briefly such as poison ivy. Many other plants can cause allergic contact dermatitis, such as certain flowers, herbs, fruits, and vegetables. Other causes of allergic contact dermatitis include: fragrances, hair dyes, metals, rubber, formaldehyde (used as a preservative in many products), and skin care products. Irritant contact dermatitis is caused when a harsh substance aggravates the skin by repeatedly contacting it. The most common example of irritant dermatitis is dry, damaged skin due to over-washing of the hands. In this case, the irritant is the water that is drying out and damaging the skin with repeated exposure. Nummular dermatitis consists of distinctive coin-shaped red plaques that are most commonly seen on the legs, hands, arms, and torso. It is more common in men than in women, and the peak age of onset is between 55 and 65. Living in a dry environment or taking frequent very hot showers can cause this condition. Atopic dermatitis, or eczema, causes the skin to itch, scale, swell, and sometimes blister. This type of eczema usually runs in families and is often associated with allergies, asthma, and stress. Defects in the skin barrier, allowing moisture out and germs in, may also come into play. Seborrheic dermatitis, called cradle cap in infants, consists of greasy, yellowish or reddish scaling on the scalp, face, or genitals. When on the face, it is typically in or near the eyebrows, or along the sides of the nose. Seborrheic dermatitis may be aggravated by stress and in adults is called dandruff. Stasis dermatitis is caused by poor circulation in the legs and can happen in people with varicose veins, congestive heart failure, or other conditions that cause chronic leg swelling. Veins in the lower legs fail to return blood efficiently, causing pooling of blood and fluid buildup and swelling. This swelling leads to skin irritation, especially around the ankles.

Additionally, compositions can be tailored and used to treat veterinary dermatoses including seborrhea, keratinization, ichthyosis, sebaceous adenitis, among other hair and dermatological disease occurring in animals. Cosmetic compositions including lotions, creams, soaps, shampoos, lip balms are that are designed for moisturizing, anti-aging, anti-wrinkle, acne treatment, rough skin treatment, and dandruff.

Without wishing to be bound by theory, the compositions of various embodiments may block inhibitory checkpoints, reducing inflammation in affected areas upon application. Various proteins have been associated with the immune checkpoint blockade including adenosine A2A receptor (A2AR), B7-H3 or CD276, MGA271, B7-H4 or VTCN1, B and T Lymphocyte Attenuator (BTLA or CD272), Cytotoxic T-Lymphocyte-Associated protein 4 (CTLA-4 or CD152), Indoleamine 2,3-dioxygenase (IDO), tryptophan 2,3-dioxygenase (TDO), Killer-cell Immunoglobulin-like Receptor (KIR), Lymphocyte Activation Gene-3 (LAG3), Programmed Death 1 (PD-1), T-cell Immunoglobulin domain and Mucin domain 3 (TIM-3), V-domain Ig suppressor of T cell activation (VISTA). The cannabidiols of embodiments may block activity of these or other immune checkpoint blockade proteins, reducing immune response and improving the symptoms of dermatitis. For example, the cannabidiols of the compositions of the invention may impact the interaction between transmembrane protein PD-1 and its ligands, PD-1 ligand 1 (PD-L1) and PD-1 ligand 2 (PD-L2) by blocking binding of PD-L1 or PD-L2 with PD-1. Inhibiting the activity of PD-1 may reduce T-cell signaling, preventing the immune response and reducing inflammation in the affected area and reducing the symptoms of dermatitis.

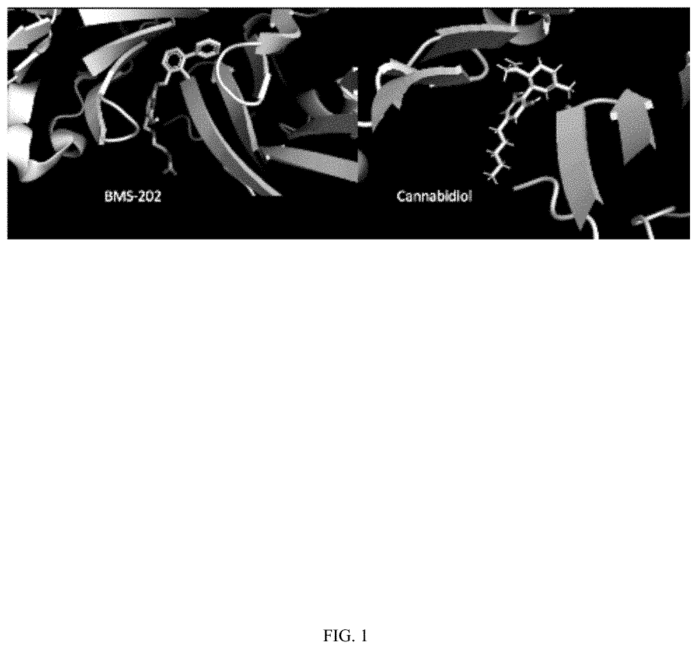

Cannabinoids may bind PD-L1 at a hydrophobic cavity created by dimerization similar to binding of known PD-1 inhibitor BMS-202. FIG. 1 compares models of PD-L1 binding of BMS-202, left panel, and cannabidiol, right panel. Three-dimensional structural similarity and the location of charged moieties suggest a similar binding orientation.

Inhibitory checkpoints like PD-1 and PD-L1 have been linked to cancer. For example, cancer-mediated upregulation of PD-L1 on the cell surface may inhibit T cells that might otherwise attack a tumor or other tumorigenic tissue. Thus, the compositions of various embodiments can be used as anti-cancer agents to block the interaction PD-1 or PD-L1, allowing T-cells to attack the tumors or tumorigenic tissue. In some embodiments, the cancers may be one or more of melanoma, lung cancer, pancreatic cancer, kidney cancer and Hodgkin's lymphoma, for example.

In various embodiments, the cannabidiol containing compositions described above may include one or more additional immune checkpoint blockade inhibitors or the cannabidiol containing compounds of the invention may be administered in combination with one or more additional immune checkpoint blockade inhibitors. Additional checkpoint blockade inhibitors include, for example, an IgG4 PD1 antibody such as, for example, antibody BGB-A317, Nivolumab, or Pembrolizumab, a PD-L1 inhibitor, such as, for example, atezolizumab, avelumab, or and durvalumab, antibodies that block the immune checkpoint molecule CTLA-4 such as, for example, ipilimumab, therapeutic agents that target an intrinsic checkpoint blockade, such as, for example, the gene encoding Cytokine-inducible SH₂-containing protein (CISH). The amount of immune checkpoint blockade inhibitor in the compositions may be from about 0.01% to about 5% (w/w), relative to the total amount of the composition, from about 0.1% to about 1% (w/w), relative to the total amount of the composition, or any range or individual concentration encompassed by these example ranges.

The cannabinoids discussed above can be combined with various additional agents to improve efficacy of treatment, aid in delivery of the cannabinoid, or treat additional symptoms associated with the dermatological disease.

For example, in some embodiments, the compositions may include one or more bioenhancers. Bioenhancers include any compound or composition that aids in the transport of another compound across epithelial membranes. Bioenhancers include P-glycoprotein inhibitors, compounds that reverse P-glycoprotein-mediated efflux, limit metabolism of active agents, increase gastric emptying time and intestinal motility, reduce degradation of the active agent by hydrochloric acid, modify cell membrane permeability, produce a cholagogue effect, modify the bioenergetics and thermogenic properties of the active agent, suppress first pass metabolism, and inhibit metabolizing enzymes, stimulate gamma glutamyl transpeptidase, enhance the uptake of amino acids, and the like and combinations thereof. In some embodiments, the bioenhancers may be herbal or neutraceutical bioenhancers. Examples of bioenhancers encompassed by the invention include piperine, quercetin, genistein, naringin, sinomenine, glycyrrhizin, nitrile glycosides cuminum cyminum, zingiver officinale, lysergol, allium sativum, aloe vera, and the like and combinations thereof. In some embodiments, the bioenhancers may be liposomes, microspheres, nanoparticles, transferosomes, ethosomes, nanoemulsions, microemulsions, lipid based systems, polymeric micelle formulations, ketoprofen-loaded solid lipid nanoparticles, and the like, which can be made from beeswax, carnauba wax, or other natural waxes and solid lipids, and combinations thereof. In some embodiments, the bioenhancers may be liposomal enhancers such as, for example, Ginkgo biloba lipid-based systems, silybin lipid-based systems, ginseng lipid-based systems, hawthorn lipid-based systems, quercetin lipid-based systems, curcumin lipid-based systems, and the like and combinations thereof. In further embodiments, the bioenhancers may be capsaicin transferosomes, colchicine tranferosomes, vincristine tranferosomes, and the like and combinations thereof, which may find particular use as natural skin penetration agents.

The amount of bioenhancer in the compositions may be from about 0.05% to about 20% (wt/wt), relative to the total amount of the composition or in some embodiments, from about 0.1% to about 10% (wt/wt), relative to the total weight of the composition, from about 0.1% to about 5% (w/w), relative to the total amount of the composition, from about 0.1% to about 2% (w/w), relative to the total amount of the composition, or any range or individual concentration encompassed by these example ranges.

For example, piperine enhances bioavailability by modulating DNA receptor binding and cell signal transduction, while inhibiting efflux pumps that remove the active agent from cells. This inhibits drug metabolizing enzymes and stimulates absorption by stimulating gut amino acid transporters and inhibiting cellular pumps responsible for drug elimination from cells and intestinal production of glucuronic acid. Piperine also increases the absorption of the active agent in the gastrointestinal tract and inhibits enzymes responsible drug metabolism especially in the liver during first pass metabolism such as hepatic arylhydrocarbon hydrolase and UDP-glucuronyltransferase activities. Piperine modifies the rate of glucuronidation by lowering the endogenous UDP-glucuronic acid content and also by inhibiting transferase activity. Piperine inhibits P-glycoprotein and cutochrome P450 3A4, also CYP1A1, CYP1B1, CYP1B2, CYP2E1, CYP3A4, among others and makes target receptors more responsive to drugs, acting as receptors for drug molecules, increasing GIT vasculature by vasodilation to increase the absorption of drugs, modulation of cell membrane dynamics which increases transport of drugs across the cell membranes.

In some embodiments, the compositions may further include hydrocortisone or any steroid within Groups I to VII in the US classification system. Group I steroids include, but are not limited to, clobetasol propionate, betamethasone dipropionate, halobetasol, and diflorasone diacetate. Group II steroids include, but are not limited to, fluocinonide, halcinonide, amcinonide, and desoximetasone. Group III steroids include, but are not limited to, triamcinolone acetonide, mometasone furoate, fluticasone propionate, betamethasone dipropionate, and halometasone. Group IV steroids include, but are not limited to, fluocinolone acetonide, hydrocortisone valerate, hydrocortisone butyrate, flurandrenolide, triamcinolone acetonide, and mometasone furoate. Group V steroids include, but are not limited to, fluticasone propionate, desonide, fluocinolone acetonide, and hydrocortisone valerate. Group VI steroids include, but are not limited to, alclometasone dipropionate, triamcinolone acetonide, fluocinolone acetonide, and desonide. Group VII steroids include, but are not limited to, hydrocortisone (2.5%) and hydrocortisone (1%). The amount of hydrocortisone or steroid in the compositions may be from about 0.01% to about 5% (w/w), relative to the total amount of the composition or in some embodiments, from about 0.1% to about 1% (w/w), relative to the total amount of the composition, or any range or individual concentration encompassed in these example ranges.

In some embodiments, the compositions may further include an anti-inflammatory compound such as methotrexate, tofacitinib, 6-mercaptopurine, azathioprine sulphasalazine, mesalazine, olsalazine chloroquinine/hydroxychloroquinine, penicillamine, aurothiomalate (intramuscular and oral), azathioprine, cochicine, corticosteroids (oral, inhaled, and local injection), a beta-2 adrenoreceptor agonist (salbutamol, terbutaline, salmeteral), xanthine (theophylline, aminophylline), cromoglycate, nedocromil, ketotifen, ipratropium and oxitropium, cyclosporin, FK506, rapamycin, mycophenolate mofetil, leflunomide, an NSAID (e.g. ibuprofen), a corticosteroid (e. g. prednisolone), a phosphodiesterase inhibitor, an adensosine agonist, an antithrombotic agent, a complement inhibitor, an adrenergic agent, an agent that interferes with signalling by proinflammatory cytokines such as TNF or IL-1 (e.g., a NIK, IKK, p38 or MAP kinase inhibitor), an IL-1 converting enzyme inhibitor, a T-cell signalling inhibitor (e.g. a kinase inhibitor), a metalloproteinase inhibitor, sulfasalazine, a 6-mercaptopurine, an angiotensin converting enzyme inhibitor, a soluble cytokine receptor (e.g. soluble p55 or p75 TNF receptors and the derivatives p75TNFRigG (etanercept) and p55TNFRigG (Lenercept), siL-1RI, siL-1RII, siL-6R), an antiinflammatory cytokine (e.g. IL-4, IL-1 0, IL-11, IL-13 and TGF), celecoxib, folic acid, hydroxychloroquine sulfate, rofecoxib, etanercept, infliximab, adalimumab, certolizumab, tocilizumab, abatacept, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone HCl, hydrocodone bitartrate/apap, diclofenac sodium/misoprostol, fentanyl, anakinra, tramadol HCl, salsalate, sulindac, cyanocobalamin/fa/pyridoxine, acetaminophen, alendronate sodium, prednisolone, cortisone, betamethasone, morphine sulfate, lidocaine hydrochloride, indomethacin, glucosamine sulf/chondroitin, amitriptyline HCl, sulfadiazine, oxycodone HCV acetaminophen, olopatadine HCl misoprostol, naproxen sodium, omeprazole, cyclophosphamide, rituximab, IL-1 TRAP, MRA, CTLA4-IG, IL-18 BP, anti-IL-12, Anti-IL1S, BIRB-796, SCIO-469, VX-702, AMG-548, VX-740, Roflumilast, IC-485, CDC-801, S1PI agonists (such as FTY720), a PKC family inhibitor (e.g. Ruboxistaurin or AEB-071) or Mesopram, budenoside; epidermal growth factor; a corticosteroid; cyclosporin, sulfasalazine; an aminosalicylate; 6-mercaptopurine; azathioprine; metronidazole; a lipoxygenase inhibitor; mesalamine; olsalazine; balsalazide; an antioxidant; a thromboxane inhibitor; an IL-1 receptor antagonist; an anti-IL-1 monoclonal antibody; an anti-IL-6 monoclonal antibody; a growth factor; an elastase inhibitor; a pyridinyl-imidazole compound; an antibody to or antagonist of other human cytokines or growth factors (e.g. TNF, LT, IL-1, IL-2, IL-6, IL-7, IL-8, IL-12, IL-15, IL-16, IL-23, EMAP-II, GM-CSF, FGF, and PDGF); a cell surface molecule (e.g. CD2, CD3, CD4, CD8, CD25, CD28, CD30, CD40, CD45, CD69, or CD90 or their ligands); methotrexate; cyclosporine; FK506; rapamycin; mycophenolate mofetil; leflunomide; an NSAID (e.g. ibuprofen); a corticosteroid (e.g. prednisolone); a phosphodiesterase inhibitor; an adenosine agonist; an antithrombotic agent; a complement inhibitor; an adrenergic agent; an agent that interferes with signalling by proinflammatory cytokines such as TNF 5 or IL-1 (e.g. a NIK, IKK, or MAP kinase inhibitor); an IL-1 converting enzyme inhibitor; a TNF converting enzyme inhibitor; a T-cell signalling inhibitor such as kinase inhibitors; a metalloproteinase inhibitor; sulfasalazine; azathioprine; a 6-mercaptopurine; an angiotensin converting enzyme inhibitor; a soluble cytokine receptor (e.g. soluble p55 or p75 TNF receptors, siL-1RI, siL-1RII, siL-6R), an antiinflammatory cytokine (e.g. IL-4, IL-1 0, IL-11, IL-13 or TGF), therapeutic agents that target an intrinsic checkpoint blockade, such as, for example, the gene encoding Cytokine-inducible SH₂-containing protein (CISH), antibody BGB-A317, Nivolumab, or Pembrolizumab, atezolizumab, avelumab, durvalumab, ipilimumab, and the like and combinations thereof. The amount of anti-inflammatory in the composition may be from about 0.01% to about 5% (w/w), relative to the total amount of the composition, or in some embodiments, from about 0.1% to about 1% (w/w), relative to the total amount of the composition, or any range or individual concentration encompassed by these example ranges.

In some embodiments, the compositions may further include an antibiotic. The antibiotic compound is not particularly limited, and antibacterial, antifungal, antiprotozoal, and other antimicrobial agents. In certain embodiments, the antibiotic may include, for example, ampicillin, bacampicillin, carbenicillin indanyl, mezlocillin, piperacillin, ticarcillin, amoxicillin-clavulanic acid, ampicillin-sulbactam, benzylpenicillin, cloxacillin, dicloxacillin, methicillin, oxacillin, penicillin G, penicillin V, piperacillin tazobactam, ticarcillin clavulanic acid, nafcillin, procaine penicillin, cefadroxil, cefazolin, cephalexin, cephalothin, cephapirin, cephradine, cefaclor, cefamandol, cefonicid, cefotetan, cefoxitin, cefprozil, ceftmetazole, cefuroxime, loracarbef cefdinir, ceftibuten, cefoperazone, cefixime, cefotaxime, cefpodoxime proxetil, ceftazidime, ceftizoxime, ceftriaxone, cefepime, azithromycin, clarithromycin, clindamycin, dirithromycin, erythromycin, lincomycin, troleandomycin, cinoxacin, ciprofloxacin, enoxacin, gatifloxacin, grepafloxacin, levofloxacin, lomefloxacin, moxifloxacin, nalidixic acid, norfloxacin, ofloxacin, sparfloxacin, trovafloxacin, oxolinic acid, gemifloxacin, perfloxacin, imipenem-cilastatin, meropenem, aztreonam, and the like and combinations thereof. Antifungal agent antibiotics include, for example, amphotericin B, candicidin, filipin, hamycin, natamycin, nystatin, rimocidin, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, fluconazole, isavuconazole, itraconazole, posaconazole, ravuconazole, terconazole, voriconazole, abafungin, amorolfin, butenafine, naftifine, terbinafine, anidulafungin, caspofungin, micafungin, benzoic acid, ciclopirox, flucytosine, griseofulvin, haloprogin, tolnaftate, undecylenic acid, crystal violet, balsam of Peru, and the like and combinations thereof. The amount of the antibiotic in the compositions may be from about 0.01% to about 5% (wt./w), relative to the total amount of the composition, or in some embodiments, from about 0.1% to about 1% (w/w), relative to the total amount of the composition, or any range or individual composition encompassed by these example ranges.

In some embodiments, the compositions may further include an antiseptic compound. The antiseptic compound is not particularly limited, and in some embodiments may include, for example, iodine, manuka honey, octenidine dihydrochloride, phenol, polyhexanide, sodium chloride, sodium hypochlorite, calcium hypochlorite, sodium bicarbonate, methyl paraben, and sodium dehydroacetate. The amount of the antiseptic in the compositions may be from about 0.01% to about 5% (w/w), relative to the total amount of the composition, or in some embodiments, from about 0.1% to about 1%, relative to the total amount of the composition, or any range or individual concentration encompassed by these example ranges.

In some embodiments, the compositions may further include an anti-acne compound. The anti-acne agent is not limited and includes, for example, salicylic acid, benzoyl peroxide, and the like and combinations thereof. The amount of the anti-acne compound in the compositions may be from about 0.01% to about 5% (w/w), relative to the total amount of the composition, or in some embodiments, from about 0.1% to about 1% (w/w), relative to the total amount of the composition, or any range or individual concentration encompassed by these example ranges.

In some embodiments, the compositions may further include humectant, which can also be referred to as a soothing, smoothing, moisturizing, or protective agent. The humectant is not particularly limited and includes, for example, calamine, dodecylsulphate, sodium lauryl sulphate (SLS), a polyoxyethylene ester of polysorbitan, such as monooleate, monolaurate, monopalmitate, monostearate esters, esters of sorbitan, polyoxyethylenes ethers, sodium dioctylsulphosuccinate (DOSS), lecithin, and sodium docusate. Sodium lauryl sulphate and calamine are the most preferred humectants. The amount of the humectant in the compositions may be from about 0.01% to about 5% (w/w), relative to the total amount of the composition, or in some embodiments, from about 0.1% to about 1% (w/w), relative to the total amount of the composition, or any range or individual composition encompassed by these example ranges.

In some embodiments, the compositions may further include a UV-absorbing compound, which can be referred to as a sunscreen agent. The UV-absorbing compound is not particularly limited and includes, for example, glyceryl PABA, padimate 0, roxadimate, dioxybenzone, oxybenzone, sulisonbenzone, octocrylene, octyl methoxycinnamate, ethoxyethyl p-methoxycinnamate, homomenthyl salicylate, ethylhexyl salicylate, trolamine salicylate, avobenzone, ecamsule, ensulizole, bemotrizinol, bisoctrizole, and the like and combinations thereof. The amount of the UV-absorbing compound in the compositions may be from about 0.01% to about 5%, relative to the total amount of the composition or in some embodiments, from about 0.1% to about 1% (w/w), relative to the total amount of the composition, or any range or individual concentration encompassed by these example ranges.

In some embodiments, the compositions may further include analgesic agent. The analgesic agent is not particularly limited and includes, for example, methyl salicylate, codeine, morphine, methadone, pethidine, buprenorphine, hydromorphine, levorphanol, oxycodone, fentanyl, a non-steroidal anti-inflammatory drug, and the like and combinations thereof. The amount of the analgesic agent in the compositions may be from about 0.01% to about 5% (w/w), relative to the total amount of the composition or in some embodiments, from about 0.1% to about 1% (w/w), relative to the total amount of the composition, or any range or individual concentration encompassed by these example ranges.

In some embodiments, the compositions may further include anti-viral compound. The anti-viral compound is not particularly limited and includes, for example, acyclovir, famciclovir, penciclovir, valacyclovir, trifluridine, docosanol, amantadine, rimantadine, oseltamivir, and zanamivir. The amount of the anti-viral compound in the compositions may be from about 0.01% to about 5% (w/w), relative to the total amount of the composition or in some embodiments, from about 0.1% to about 1% (w/w), relative to the total amount of the composition, or any range or individual concentration encompassed by these example ranges.

The oils and other active agents discussed above can be supplemented with any of the additives discussed below and can be incorporated into creams, lotions, salves, liniments, ointments, gels, pastes, tonics, tinctures, unguents, soaps, shampoos, orals, pills, tablets, capsules, and lip balms discussed below. Thus, the form of the compositions of the invention is not limited.

Creams refer to semi-solid emulsions of oil and water in approximately equal proportions. They are divided into two types: oil-in-water (O/W) creams, composed of small droplets of oil dispersed in a continuous phase; and water-in-oil (W/O) creams, composed of small droplets of water dispersed in a continuous oily phase. Creams can provide a barrier to protect the skin. This may be a physical barrier or a chemical barrier as with UV-absorbing compounds. To aid in the retention of moisture (especially water-in-oil creams), creams are usually used for a variety of purposes including cleansing, emollient effects, and as a vehicle for drug substances such as local anesthetics, anti-inflammatories (NSAIDs or corticosteroids), hormones, antibiotics, antifungals or counter-irritants.

Liniments or balms are topical compositions that are of a similar viscosity to lotions and less viscous than an ointment or cream. Liniments are generally applied with friction by rubbing the liniment into the skin. Liniments typically are formulated from alcohol, acetone, or similar quickly evaporating solvents and may contain counterirritant aromatic chemical compounds such as methyl salicylate, benzoin resin, or capsaicin.

Ointments are compositions in which oil and water are provided in a ratio of from 7:1 to 2:1, from 5:1 to 3:1, or 4:1. Ointments are generally formulated using oils, waxes, water, alcohols, petroleum products, water, and other agents to prepare compositions with various viscosities and solvent properties. Commonly used compositions include oleaginous base (White Ointment), absorption base, W/O emulsion base (Cold Cream type base), O/W emulsion base (Hydrophilic Ointment), water soluble base, in addition to others. These preparations are used to dissolve or suspend substances or products with medicinal or cosmetic value.

Lotions are low- to medium-viscosity topical preparations. Most lotions are oil-in-water emulsions containing an emulsifier such as cetyl alcohol to prevent separation of these two phases. Lotions can include fragrances, glycerol, petroleum jelly, dyes, preservatives, proteins and stabilizing agents.

In some embodiments, the compositions can be in the form of a soap, which are compositions that comprise a salt of a fatty acid. Soaps are mainly used as surfactants for washing, bathing, and cleaning, but they are also used in textile spinning and are important components of lubricants. Soaps for cleansing are usually obtained by treating vegetable or animal oils and fats with a strongly alkaline solution. Fats and oils are composed of triglycerides; three molecules of fatty acids are attached to a single molecule of glycerol. The alkaline solution, which is often called lye (although the term “lye soap” refers almost exclusively to soaps made with sodium hydroxide), is believed to promote a chemical reaction known as saponification. In saponification, the fats are first hydrolyzed into free fatty acids, which then combine with the alkali to form crude soap. Glycerol (glycerine) is usually liberated and is either left in or washed out and recovered as a useful byproduct, depending on the process employed.

In some embodiments, the compositions can be in the form of a shampoo, which is a hair care product used for the removal of oils, dirt, skin particles, dandruff, environmental pollutants and other contaminant particles that gradually build up in hair. A goal may be to remove the unwanted build-up without stripping out so much sebum as to make hair unmanageable.

Another embodiment of the present invention is a method of making the composition in the form of a cream, which comprises (i) dispersing lake/powder into mineral oil or silicone oil to obtain an oil phase; (ii) dispersing an emulsifier, a thickener; and a stabilizer into water in a separate vessel to obtain an aqueous phase; (iii) blending the oil phase and the aqueous phase to form an emulsion; and (iv) dispersing an active ingredient such as a Cannabis derived botanical drug product into at least one of the oil phase, the aqueous phase, and the emulsion. In some embodiments, the method further comprises heating during at least one of (i) dispersing lake/powder into mineral oil or silicone oil to obtain an oil phase and (ii) dispersing an emulsifier, a thickener; and a stabilizer into water in a separate vessel to obtain an aqueous phase. Temperatures of this heating are not particularly limited, so long as the oil phase and the aqueous phase result from the dispersing.

Another embodiment of the present invention is a method of making the topical composition in the form of a lotion, which comprises mixing an oil phase comprising hemp oil with an emulsifier and with an aqueous phase to form a mixture and heating said mixture at a temperature of from 45 and 85° C. to form an aqueous emulsion. Emulsifiers include, but are not limited to, cetyl alcohol, stearic acid, and a mixture thereof. The water phase comprises a stabilizing agent such as VEEGUM® or CARBOPOL®.

Another embodiment of the present invention is a method of making the composition in the form of a shampoo, which comprises combining a surfactant, most often sodium lauryl sulfate and/or sodium laureth sulfate with a co-surfactant, most often cocamidopropyl betaine, in an aqueous phase and mixing the aqueous phase to form a thick, viscous liquid. Preferred methods further comprise adding other ingredients, such as salt (sodium chloride), a preservative, and fragrance, to the aqueous phase.

Another embodiment of the present invention is a method of making the composition in the form of a tincture. Tinctures are herbal extracts that provide a method for oral administration of an herbal component of components to a subject in need of treatment. Tinctures are prepared by mixing an herb or herbs or components or combinations thereof with a suitable solvent wherein a component or components of an herb or combinations thereof are extracted into a solvent in which the component or components of the herbs are reasonably soluble. Suitable tincture solvents in the present invention include pharmacologically acceptable solvent such as organic solvents, water based solvents, alcohols, and other orally administrable solvents such as, but not limited to, water, purified water, preserved water, vegetable glycerin, propylene carbonate, 3-methoxy-3-methyl-1-butanol (MMB), polyethylene glycerol, rice bran oil, and combinations thereof.

In some embodiments, the composition can be in the form of a tonic. Tonics are extracts that provide a method for oral administration of an herbal component or components to a subject in need of treatment. Tonics are prepared by mixing an herb or herbs or components or combinations thereof with a suitable solvent wherein a component or components of an herb or herbs or combinations thereof are extracted into a solvent by aid of heating, often heat necessary such that the solvent reaches its boiling temperature, in which the component or components of the herb are reasonably soluble. Suitable tonic solvents in the present invention include pharmacologically acceptable solvents such as organic solvents, water based solvents, alcohols, and other orally administrable solvents such as, but not limited to, water, purified water, preserved water, vegetable glvcerin, propylene carbonate, 3-methoxy-3methyl-1-butanol (MMB), polyethylene glycol, rice bran oil, and combinations thereof.

In some embodiments, the composition can be in the form of a tablet. Tablets are pharmaceutical oral doses of a medicament or medicaments that are formed by molding or compression. Such embodiments are comprised of the medicament or medicaments and may be further comprised of suitable excipients such as, but not limited to, diluents, binders, granulating agents, gildants, lubricants, disintegrants, sweeteners, and pigments. Tablets in the present invention may also be coated with a pigment to increase the visual appearance of the tablet, to increase the identifiability of the tablet, to increase the ease with which the tablet is orally administered, to make the tablet more easily swallowed, to control the release of the medicament or medicaments, or to make the tablet more resistant to environmental degradation factors, or a combination or combinations thereof.

In another embodiment, the composition can be in the form of a capsule. Capsules generally fall within the class of either hard-shelled capsules or soft-shelled capsules, but need not be restricted to either class. Hard shelled capsules generally, but need not necessarily, contain dry, powdered, or granular components while soft-shelled capsules primarily, but need not necessarily, contain oils or medicaments or combinations thereof.

Another embodiment of the present invention is a method of treating a dermatological disease, which comprises applying a therapeutically effective amount of the topical composition according to the present invention to skin affected with a dermatological disease. Non-limiting examples of targeted dermatological diseases include eczema, psoriasis, sunburn, contact dermatitis, poison ivy and conditions caused by other plant materials containing urushiol or related molecules, type 1 and type 2 herpes, insect bites, anal itching, vaginal itching, acne, warts and other acute and chronic dermatoses afflicting humans, and use as a topical analgesic for muscle and arthritic pain.

In some embodiments, the method may include tapering doses of cannabidiol. Thus, embodiments may include a treatment regimen in which compositions containing different concentrations of cannabidiol are administered over the course of treatment. For example, some embodiments include applying one or more doses of a first composition containing greater than 10% (w/w) cannabidiol to a subject in need of treatment then subsequently administering one or more doses of a second composition containing less cannabidiol than the first topical composition to the subject in need of treatment. For example, if the first composition contains about 20% (w/w) cannabidiol, the second composition may include 19% (w/w) or less cannabidiol. In further embodiments, the method may include administering one or more doses of a third composition containing less cannabidiol than the second composition, a fourth composition containing less cannabidiol than the third composition, and so on. In particular embodiments, the method may include administering a composition containing a maintenance dose of cannabidiol that is equal to or less than the final dosage of a dosage regimen containing two or more compositions of decreasing dosage. The maintenance dose may provide sufficient cannabidiol to reduce or eliminate potential recurrence of the dermatological disease.

### EXAMPLES

Although the present invention has been described in considerable detail with reference to certain preferred embodiments thereof, other versions are possible. Therefore, the spirit and scope of the appended claims should not be limited to the description and the preferred versions contained within this specification. Various aspects of the present invention will be illustrated with reference to the following non-limiting examples.

### Example 1

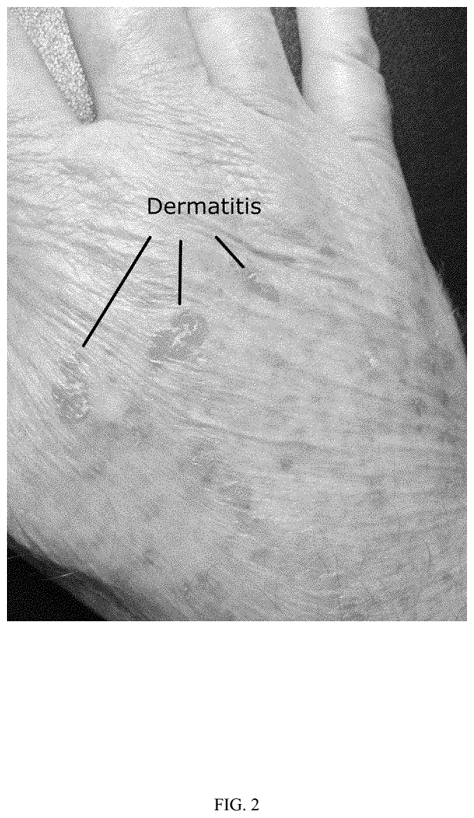

An ointment containing 1% CBDA was applied to the skin of a patient having dermatitis for one month. FIG. 1 shows the patient's hand before treatment, and FIG. 2 shows the patient's hand following one month of treatment. The affected areas in FIG. 2 are less red and puffy suggesting a reduction in inflammation, and the skin surrounding the inflamed areas are less dry and flaky. These data suggest at least an 80% reduction in the symptoms of dermatitis exhibited by the patient after one month of treatment.

### Example 2

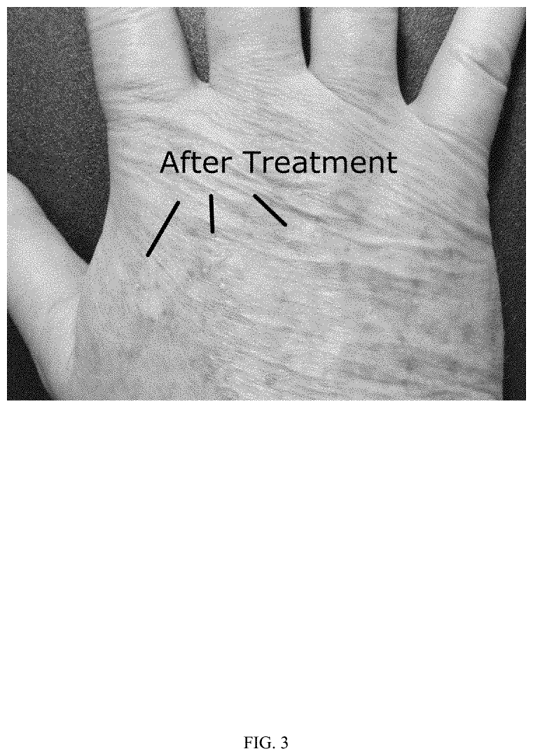

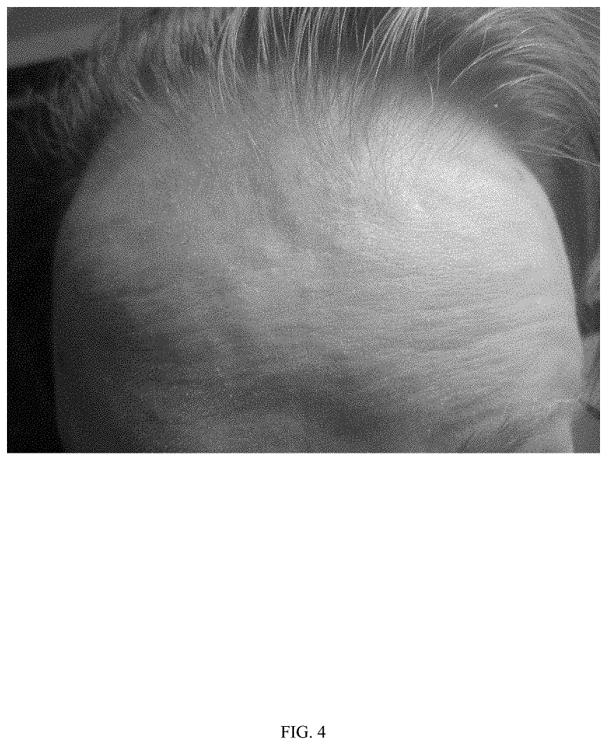

An ointment containing 5% CBDA was applied to the skin of a patient having steroid resistant seborrheic dermatitis on their forehead and scalp. FIG. 3 shows the patient's affected area prior to treatment. FIG. 4 shows the patient's affected area after 1 week of treatment. These data suggest a 100% reduction in the affected area with no recurrence after a period of 6 months.

### Example 3

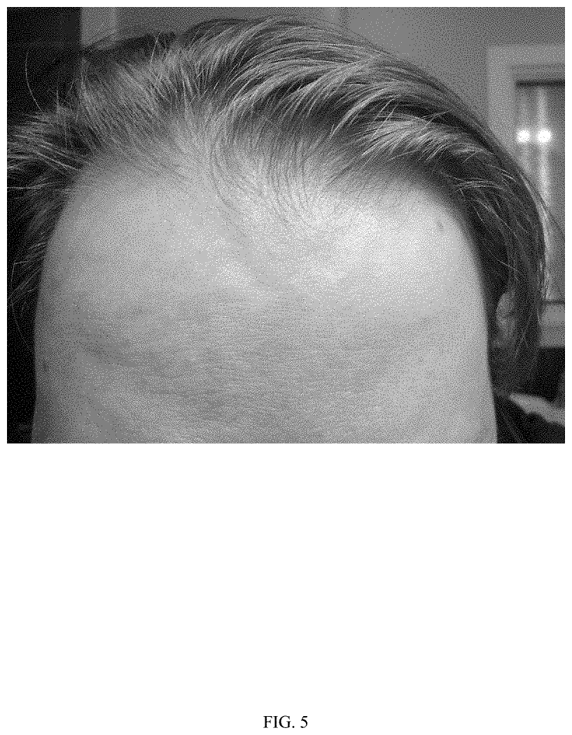

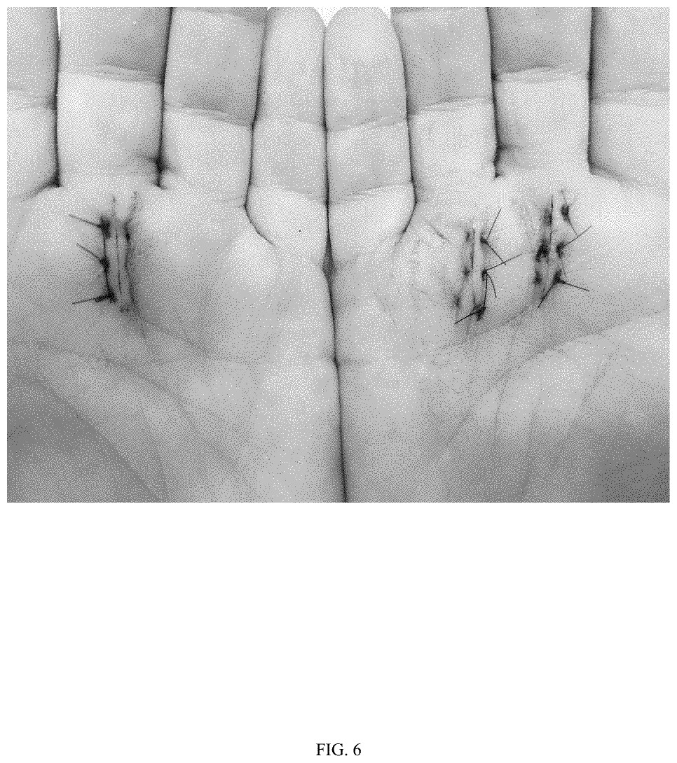

An ointment containing 1% CBDA was applied to the skin of a patient who recently had bilateral hand surgery to the left hand twice daily while the right hand received no treatment in comparison. FIG. 5 shows the patient's hands before treatment, and FIG. 6 shows the patient's hands after 4 weeks of treatment. The affected area on the left hand showed advanced healing and reduction of the appearance of scarring compared to the right hand by greater than 50%.

1. A composition comprising a cannabinoid having a concentration of about 0.5% (w/w) to about 50% (w/w), relative to the total amount of the composition and a pharmaceutically acceptable carrier, excipient, diluent, reagent, or combinations thereof.

2. The composition of claim 1, wherein the cannabinoid has a concentration of about 20% (w/w) to about 1% (w/w) cannabidiol, relative to the total amount of the composition.

3. The composition of claim 1, wherein the cannabinoid is selected from the group consisting of cannabidiol, cannabidiol isomer, cannabidiol analog, or combinations thereof.

4. The composition of claim 1, further comprising a bioenhancer having a concentration of about 0.05% wt to about 20% wt, relative to the total weight of the composition.

5. The composition of claim 4, wherein the bioenhancer comprises a P-glycoprotein inhibitor selected from the group consisting of piperine, quercetin, genistein, naringin, sinomenine, glycyrrhizin, nitrile glycosides cuminum cyminum, zingiver officinale, lysergol, allium sativum, aloe vera, and combinations thereof.

6. The composition of claim 4, wherein the bioenhancer is selected from the group consisting of liposomes, microspheres, nanoparticles, transferosomes, ethosomes, nanoemulsions, microemulsions, lipid based systems, polymeric micelle formulations, ketoprofen-loaded solid lipid nanoparticles, and the like, which can be made from beeswax, carnauba wax, or other natural waxes and solid lipids, and combinations thereof.

7. The composition of claim 4, wherein the bioenhancer is selected from the group consisting of Ginkgo biloba lipid-based systems, silybin lipid-based systems, ginseng lipid-based systems, hawthorn lipid-based systems, quercetin lipid-based systems, curcumin lipid-based systems and combinations thereof.

8. The composition of claim 4, wherein the bioenhancer is selected from the group consisting of capsaicin transferosomes, colchicine tranferosomes, vincristine tranferosomes, and combinations thereof.

9. The composition of claim 1, further comprising a humectant selected from the group consisting of calamine, dodecylsulphate, sodium lauryl sulphate (SLS), a polyoxyethylene ester of polysorbitan, such as monooleate, monolaurate, monopalmitate, monostearate esters, esters of sorbitan, polyoxyethylenes ethers, sodium dioctylsulphosuccinate (DOSS), lecithin, sodium docusate, and combinations thereof.

10. The composition of claim 9, wherein the amount of the humectant has a concentration of about 0.01 wt. % to about 5 wt. %, relative to the total weight of the composition.

11. The composition of claim 1, further comprising a steroid, anti-inflammatory, immune checkpoint blockade inhibitor, antibiotic, antiseptic, anti-acne agent, UV-absorbing compound, analgesic agent, or an antiviral compound, or combinations thereof.

12. The composition of claim 11, wherein the amount of steroid, anti-inflammatory, immune checkpoint blockade inhibitor, antibiotic, antiseptic, anti-acne agent, UV-absorbing compound, analgesic agent, or antiviral compound, or combinations thereof has a concentration of about 0.01% to about 50% (wt/wt), relative to the total amount of the composition.

13. The composition of claim 1, wherein the composition is in the form of a cream, lotion, salve, liniment, ointment, gel, paste, tonic, tincture, unguent, soap, shampoo, topical, oral, pills, tablet, capsule, lip balm, or combinations thereof.

14. A method for treating a dermatological disease, comprising administering a composition containing a cannabinoid having a concentration of about 0.5% (w/w) to about 50% (w/w) to a patient in need of treatment.

15. The method of claim 14, wherein the dermatological disease is selected from the group consisting of dermatitis and scarring.

16. The method of claim 14, wherein the concentration of the cannabinoid has a concentration in the range of about 1% to about 30% (w/w), relative to the total amount of the composition.

17. The method of claim 14, wherein the cannabinoid has a concentration in the range of about 5% to about 20% (w/w), relative to the total amount of the composition.

18. The method of claim 14, wherein the composition further comprises a bioenhancer.

19. The method of claim 18, wherein the bioenhancer has a concentration in the range of about 0.05% wt to about 20% wt, relative to the total weight of the composition.

20. The method of claim 14, wherein the composition further comprises a steroid, humectant anti-inflammatory, immune checkpoint blockade inhibitor, antibiotic, antiseptic, anti-acne agent, UV-absorbing compound, analgesic agent, or an antiviral compound, or combinations thereof.
